(19) United States
(12) Patent Application Publication
(10) Pub. No.: US 2025/0188483 A1
(43) Pub. Date: Jun. 12, 2025

(54) METHODS FOR NON-TRANSGENIC GENOME EDITING IN PLANTS

(71) Applicant: CELLECTIS, Paris (FR)

(72) Inventors: Daniel F. Voytas, Falcon Heights, MN (US); Feng Zhang, Maple Grove, MN (US); Jin Li, Ankeny, IA (US); Thomas Stoddard, St. Louis Park, MN (US); Song Luo, Chicago, IL (US)

(73) Assignee: CELLECTIS, Paris (FR)

(21) Appl. No.: 18/737,469

(22) Filed: Jun. 7, 2024

Related U.S. Application Data
(63) Continuation of application No. 17/022,421, filed on Sep. 16, 2020, now abandoned.
Application No. 17/022,421 is a continuation of application No. 14/898,208, filed on Dec. 14, 2015, now abandoned.
(60) Provisional application No. 61/835,307, filed on Jun. 14, 2013.

(51) Int. Cl. C12N 15/82; C12N 9/22

(52) U.S. Cl. CPC C12N 15/8206; C12N 9/22; C12N 15/8207; C12N 15/8213

(57) ABSTRACT

Materials and methods for creating genome-engineered plants with non-transgenic methods are provided herein.

### CROSS-REFERENCE TO RELATED APPLICATIONS

This application is a continuation of U.S. application Ser. No. 17/022,421, filed Sep. 16, 2020, which is a continuation of U.S. application Ser. No. 14/898,208, filed Dec. 14, 2015 (abandoned), which is a National Stage Application under 35 U.S.C. § 371 of PCT Application No. PCT/IB2014/062223, filed Jun. 13, 2014, which claims benefit of priority from U.S. Provisional Application Ser. No. 61/835,307, filed on Jun. 14, 2013.

### SEQUENCE LISTING

This application contains a Sequence Listing that has been submitted electronically as an XML file named “36861-0005003 SL ST26.XML.” The XML file, created on Jun. 7, 2024, is 66,887 bytes in size. The material in the XML file is hereby incorporated by reference in its entirety.

### TECHNICAL FIELD

This document relates to the field of plant molecular biology, and in particular provides materials and methods for creating genome-engineered plants with non-transgenic methods.

### BACKGROUND

Traditional plant breeding strategies have been developed over many years to introduce desirable traits into plant species such as increased drought tolerance and crop yield. Such strategies have the drawback that they typically require many successive rounds of crossing, and thus it can take many years to successfully alter a specific plant trait. With the advent of transgenic technologies it became possible to engineer plants with genomic alterations by introducing transgene constructs and thus circumvent the need for traditional plant breeding. However, these transgenic techniques also had several drawbacks. First, transgene insertion into the genome (such as that mediated by Agrobacterium tumefaciens) is largely random and can lead to multiple insertions which can cause difficulties in tracking multiple transgenes present on different chromosomes during segregation. Further, expression of the transgene can be unpredictable due to its chromosomal environment, and in many cases expression of the transgene is silenced. In addition, production of transgenic plants has proven to be a very controversial topic, with public opinion often being against the creation of such varieties—particularly where the varieties in question are crop plants that will be grown over large geographical areas and used as food for human consumption.

Methods that allow for targeted modification of the plant genome may overcome the first two of these problems, making it possible to target transgene insertions to single chromosomal sites that are conducive to gene expression, thus reducing or eliminating the possibility of multiple transgene insertions and silencing events. Targeted genome modification has been demonstrated in a number of species using engineered Zinc Finger Nucleases (ZFNs), which permit the creation of double stranded DNA break points at preselected loci and the subsequent insertion of transgenes in a targeted manner (Lloyd et al. 2005; Wright et al. 2005; Townsend et al. 2009). A variation on this technique is to simply use the ZEN to create a break point at a chosen locus and then allow repair of the DNA by NHEJ (non-homologous end joining). During this process, errors are often incorporated into the newly joined region (e.g., nucleotide deletions) and this method allows for the targeted mutagenesis of selected plant genes as well as for the insertion of transgene constructs.

### SUMMARY

The above-mentioned advances in plant genetic engineering do not necessarily allay public fears concerning the production and widespread growth of engineered plant species. A solution to this problem would therefore be a method which can precisely alter the genome of a plant in a targeted way without the use of traditional transgenic strategies. The disclosure herein provides such a solution by providing methods for targeted, non-transgenic editing of a plant genome. The methods more particularly rely on the introduction into a plant cell of sequence-specific nucleases under protein or mRNA forms, which are translocated to the nucleus and which act to precisely cut the DNA at a predetermined locus. Errors made during the repair of the cut permit the introduction of loss of function (or gain of function) mutations without the introduction into the genome of any exogenous genetic material.

In this way, genetically modified plant species can be produced that contain no residual exogenous genetic material.

Prior to development of the methods described herein, genetic modification of plant cells required the stable genomic integration of a transgene cassette for the expression in vivo of a nuclease or a DNA modifying enzyme. Such integration was typically achieved through Agrobacterium-mediated transformation of plant species. As described herein, however, consistent and reproducible genomic modification can be achieved through the introduction into a plant cell of either purified nuclease protein or mRNA encoding for such nuclease. This is an unexpected effect, because recombinant nucleases or purified mRNA were not considered to be sufficiently active to have a significant effect on plant chromosomal or organelle DNA. Further, this document provides new protocols for genomic modification, and also provides sequences and vectors suitable to practice the methods described herein, and to produce modified plant cells without introducing exogenous DNA.

This document describes methods for editing plant genomes using non-transgenic strategies. Sequence-specific nucleases (including ZFNs, homing endonucleases, TAL-effector nucleases, CRISPR-associated systems [Cas9]) are introduced into plant cells in the form of purified nuclease protein or as mRNA encoding the nuclease protein. In the case of CRISPR-associated systems [Cas9], the nuclease can be introduced either as mRNA or purified protein along with a guide RNA for target site recognition.

The functional nucleases are targeted to specific sequences, and cut the cellular DNA at predetermined loci. The DNA damage triggers the plant cell to repair the double strand break. Mistakes (e.g., point mutations or small insertions/deletions) made during DNA repair then alter DNA sequences in vivo.

Unlike conventional DNA transformation, the protein or RNA-based genome editing strategies described herein specifically modify target nucleic acid sequences and leave no footprint behind. Since no foreign DNA is used in these methods, this process is considered to be non-transgenic plant genome editing.

In one aspect, this document features a method for targeted genetic modification of a plant genome without inserting exogenous genetic material. The method can include (i) providing a plant cell that contains an endogenous gene to be modified, (ii) obtaining a sequence-specific nuclease containing a sequence recognition domain and a nuclease domain; (iii) transfecting the plant cell with the sequence-specific nuclease, and (iv) inducing one or more double stranded DNA breaks (DSB) in the genome, to produce a plant cell or cells having a detectable targeted genomic modification without the presence of any exogenous genetic material in the plant genome. The DSB can be repaired by non-homologous end joining (NHEJ).

The sequence-specific nuclease can be a TAL effector-nuclease, a homing endonuclease, a zinc finger nuclease (ZFN), or a CRISPR-Cas9 endonuclease. The sequence-specific nuclease can be delivered to the plant cell in the form of a purified protein, or in the form of purified RNA (e.g., an mRNA).

The sequence-specific nuclease can further contain one or more subcellular localization domains. The one or more subcellular localization domains can include an SV40 nuclear localization signal, an acidic M9 domain of hnRNPA1, a PY-NLS motif signal, a mitochondrial targeting signal, or a chloroplast targeting signal. The sequence-specific nuclease can further contain one or more cell penetrating peptide domains (CPPs). The one or more CPPs can include a transactivating transcriptional activator (Tat) peptide or a Pep-1 CPP domain.

The sequence-specific nuclease can be co-transfected with one or more plasmids encoding one or more exonucleases. The one or more exonucleases can include a member of the TREX exonuclease family (e.g., TREX2).

The endogenous gene to be modified can be an acetolactate synthase gene (e.g., ALS1 or ALS2), or a vacuolar invertase gene (e.g., the potato (Solanum tuberosum) vacuolar invertase gene (VInv).

The plant cell can be from a field crop species of alfalfa, barley, bean, corn, cotton, flax, pea, rape, rice, rye, safflower, sorghum, soybean, sunflower, tobacco, wheat. The plant cell can be from the genus Nicotiana, or from the species Arabidopsis thaliana.

Transfection can be effected through delivery of the sequence-specific nuclease into isolated plant protoplasts. For example, transfection can be effected delivery of the sequence-specific nuclease into isolated plant protoplasts using polyethylene glycol (PEG) mediated transfection, electroporation, biolistic mediated transfection, sonication mediated transfection, or liposome mediated transfection.

Induction of one or more double stranded DNA breaks in the genome can be followed by repair of the break or breaks through a homologous recombination mechanism.

This document also features a transformed plant cell obtainable according to the methods provided herein, as well as a transformed plant containing the plant cell.

In another aspect, this document features a kit for targeted genetic modification of a plant genome without inserting exogenous genetic material. The kit can include (i) one or more sequence-specific nucleases in protein or mRNA format, (ii) one or more plant protoplasts or whole cultured plant cells, and optionally (iii) one or more DNA plasmid vectors encoding one or more exonucleases.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a diagram depicting a structural organization of the sequence-specific nucleases used for genome engineering. The nucleases contain domains that function to enable cell penetration, sub-cellular protein localization, DNA sequence recognition, and DNA cleavage.

FIG. 2 is a picture showing SDS-PAGE of transcription activator-like effector endonucleases (TALEN™) produced in E. coli. ALS2T1L and ALS2T1R are TALEN™s that target a site in the Nicotiana benthamiana ALS2 gene. VInv7 is a compact TALEN™ (CT) that targets the potato VInv gene.

FIG. 3 is a picture of an agarose gel showing in vitro activity of purified TALEN™s targeting the N. benthamiana ALS2 gene. A PCR product was generated that contains the target site for the ALS2T1 TALEN™s. The PCR product was incubated without (−) or with (+) the purified ALS2T1L and ALS2T1R proteins. Only in the presence of the two proteins was the PCR product cleaved. As a negative control, the purified ALS2T1L and ALS2T1R proteins were incubated with a PCR product from the ALS1 gene; no cleavage was observed.

FIG. 4 is a table showing the activity of I-SceI on episomal targets when delivered to plant cells as a protein.

FIG. 5 is a table showing activity of I-SceI activity on a chromosomal site when delivered to plant cells as a protein alone or in combination with TreX. The number in the total number of 454 sequencing reads used for this analysis is indicated in parentheses in column 2.

FIG. 6 is a sequence alignment showing examples of I-SceI-induced mutations in a transgenic N. tabacum line that contains an integrated I-SceI recognition site. The top line (SEQ ID NO:8) indicates the DNA sequence of the recognition site for I-SceI (underlined). The other sequences (SEQ ID NOS:9 to 18) show representative mutations that were induced by imprecise non-homologous end-joining (NHEJ).

FIG. 7 is a graph summarizing TALEN™ ALS2T1 mutagenesis activity after transformation into plant cells in different forms (DNA or protein) or treatment combinations.

FIG. 8 is a sequence alignment showing examples of TALEN™ ALS2T1 induced mutations in the N. benthamiana ALS2 gene. The top line (SEQ ID NO:19) shows the DNA sequence of the recognition site for ALS2T1 (underlined). The other sequences (SEQ ID NOS:20 to 31) show representative mutations that were induced by imprecise non-homologous end-joining (NHEJ).

FIG. 9 is a diagram showing the structural organization of a sequence-specific nuclease as used for in vitro mRNA production. The nuclease construct contains a T7 promoter, a nuclease ORF, and a 121-bp polyA tail.

FIG. 10 is a graph plotting the cleavage activity of I-CreI mRNA delivered to plant protoplasts in a YFP-based SSA assay. A SSA target plasmid together with p35S-I-CreI or I-CreI mRNA was co-delivered to tobacco protoplasts via PEG-mediated transformation. Twenty four hours after transformation, the protoplasts were subjected to flow cytometry to quantify the number of YFP-positive cells.

FIG. 11 is the target sequence (SEQ ID NO:32) for the XylT_T04 TALEN™ in N. benthamiana.

FIG. 12 is a table recapitulating the 454 pyro-sequencing data for delivery of Xyl_T04 TALEN™ mRNA to tobacco protoplasts. The numbers in parenthesis in column 3 are the total number of sequencing reads obtained *: NHEJ mutagenesis frequency was obtained by normalizing the percentage of 454 reads with NHEJ mutations to the protoplast transformation efficiency. The total number of 454 sequencing reads used for this analysis is indicated in parentheses. **: Negative controls were obtained from protoplasts transformed only by the YFP-coding plasmid.

FIG. 13 is a sequence alignment showing examples of mutations induced by XylT_T04 mRNA in N. benthamiana at the XylT1 (SEQ ID NOS:33 to 43) and XylT2 (SEQ ID NOS:44 to 54) genes.

### DETAILED DESCRIPTION

This document provides new strategies for editing plant genomes to generate non-transgenic plant material. The methods provided herein are carried out using a nuclease designed to recognize specific sequences in any site of the plant genome.

In one aspect, this document relates to a method for targeted genetic modification of a plant genome without inserting exogenous genetic material comprising one or several of the following steps:
i) providing a plant cell which comprises an endogenous gene to be modified;
ii) obtaining a sequence-specific nuclease comprising a sequence recognition domain and a nuclease domain;
iii) transformation of the plant cell with said sequence-specific nuclease, and
iv) induction of one or more double stranded DNA breaks in the genome;

This method aims at producing a plant cell or cells having a detectable targeted genomic modification, preferably without the presence of any exogenous genetic material in the plant genome.

Induction of double stranded breaks in the genome generally leads to repair of the breaks by non homologous end joining (NHEJ), which favours deletion, correction or insertion of genetic sequences into the genome of the plant cell obtained by the method.

After the coding sequence for the nuclease has been synthesized and cloned into an expression vector, nuclease protein or mRNA is produced and purified. To improve efficacy, cell penetrating peptides (CPP) can be added to improve cell membrane permeability for small molecules (drugs), proteins and nucleic acids (Mäe and Langel, 2006; US 2012/0135021, US 2011/0177557, U.S. Pat. No. 7,262,267). Sub-cellular localization peptides can also be added to direct protein traffic in cells, particularly to the nucleus (Gaj et al. 2012; US 20050042603).

In some embodiments, a protein with exonuclease activity, such as, for example, Trex (WO 2012/058458) and/or Tdt (Terminal deoxynucleotidyl transferase) (WO 2012/13717), is co-delivered to the plant cell for increasing sequence-specific nuclease induced mutagenesis efficiency. Trex2 (SEQ ID NO:6) has shown to be particularly effective by increasing mutagenesis as described herein. Using Trex2 expressed as a single polypeptide chain (SEQ ID NO:7) was even more effective.

Purified nucleases are delivered to plant cells by a variety of means. For example, biolistic particle delivery systems may be used to transform plant tissue. Standard PEG and/or electroporation methods can be used for protoplast transformation. After transformation, plant tissue/cells are cultured to enable cell division, differentiation and regeneration. DNA from individual events can be isolated and screened for mutation.

In some embodiments, the sequence-specific nuclease is a TAL-effector nuclease (Beurdeley et al., 2013). It is also envisaged that any type of sequence-specific nuclease may be used to perform the methods provided herein as long as it has similar capabilities to TAL-effector nucleases. Therefore, it must be capable of inducing a double stranded DNA break at one or more targeted genetic loci, resulting in one or more targeted mutations at that locus or loci where mutation occurs through erroneous repair of the break by NHEJ or other mechanism (Certo et al., 2012). Such sequence-specific nucleases include, but are not limited to, ZFNs, homing endonucleases such as I-SceI and I-CreI, restriction endonucleases and other homing endonucleases or TALEN™s. In a specific embodiment, the endonuclease to be used comprises a CRISPR-associated Cas protein, such as Cas9 (Gasiunas et al., 2012).

The sequence-specific nuclease to be delivered may be either in the form of purified nuclease protein, or in the form of mRNA molecules which can are translated into protein after transfection. Nuclease proteins may be prepared by a number of means known to one skilled in the art, using available protein expression vectors such as, but not limited to, pQE or pET. Suitable vectors permit the expression of nuclease protein in a variety of cell types (E. coli, insect, mammalian) and subsequent purification. Synthesis of nucleases in mRNA format may also be carried out by various means known to one skilled in the art such as through the use of the T7 vector (pSF-T7) which allows the production of capped RNA for transfection into cells.

In some embodiments, the mRNA is modified with optimal 5′ untranslated regions (UTR) and 3′ untranslated regions. UTRs have been shown to play a pivotal role in post-translational regulation of gene expression via modulation of localization, stability and translation efficiency (Bashirullah, 2001). As noted above, mRNA delivery is desirable due to its non-transgenic nature; however, mRNA is a very fragile molecule, which is susceptible to degradation during the plant transformation process. Utilization of UTRs in plant mRNA transformations allow for increased stability and localization of mRNA molecules, granting increased transformation efficiency for non-transgenic genome modification.

In some embodiments, the engineered nuclease includes one or more subcellular localization domains, to allow the efficient trafficking of the nuclease protein within the cell and in particular to the nucleus (Gaj. et al., 2012; US 2005/0042603). Such a localization signal may include, but is not limited to, the SV40 nuclear localization signal (Hicks et al., 1993). Other, non-classical types of nuclear localization signal may also be adapted for use with the methods provided herein, such as the acidic M9 domain of hnRNP A1 or the PY-NLS motif signal (Dormann et al., 2012). Localization signals also may be incorporated to permit trafficking of the nuclease to other subcellular compartments such as the mitochondria or chloroplasts. Guidance on the particular mitochondrial and chloroplastic signals to use may be found in numerous publications (see, Bhushan S. et al., 2006) and techniques for modifying proteins such as nucleases to include these signals are known to those skilled in the art.

In some embodiments, the nuclease includes a cell-penetrating peptide region (CPP) to allow easier delivery of proteins through cell membranes (Mäe and Langel, 2006; US 2012/0135021, US 2011/0177557, U.S. Pat. No. 7,262,267). Such CPP regions include, but are not limited to, the transactivating transcriptional activator (Tat) cell penetration peptide (Lakshmanan et al., 2013, Frankel et al., 1988). It is envisaged that other CPP's also may be used, including the Pep-1 CPP region, which is particularly suitable for assisting in delivery of proteins to plant cells (see, Chugh et al., 2009).

In some embodiments, one or more mutations are generated in the coding sequence of one of the acetolactate synthase (ALS) genes ALS1 or ALS2, or one or more mutations are generated in the vacuolar invertase (VInv) gene. In a further aspect of these embodiments, the mutation may be any transition or transversion which produces a non-functional or functionally-reduced coding sequence at a predetermined locus. It is generally envisaged that one or more mutations may be generated at any specific genomic locus using the methods described herein.

In some embodiments, the plant species used in the methods provided herein is N. benthamiana, although in a further aspect the plant species may be any monocot or dicot plant, such as (without limitation) Arabidopsis thaliana; field crops (e.g., alfalfa, barley, bean, corn, cotton, flax, pea, rape, rice, rye, safflower, sorghum, soybean, sunflower, tobacco, and wheat); vegetable crops (e.g., asparagus, beet, broccoli, cabbage, carrot, cauliflower, celery, cucumber, eggplant, lettuce, onion, pepper, potato, pumpkin, radish, spinach, squash, taro, tomato, and zucchini); fruit and nut crops (e.g., almond, apple, apricot, banana, blackberry, blueberry, cacao, cherry, coconut, cranberry, date, fajoa, filbert, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut, and watermelon); and ornamentals (e.g., alder, ash, aspen, azalea, birch, boxwood, camellia, carnation, chrysanthemum, elm, fir, ivy, jasmine, juniper, oak, palm, poplar, pine, redwood, rhododendron, rose, and rubber).

In some embodiments, the protein or mRNA encoding the nuclease construct is delivered to the plant cells via PEG-mediated transformation of isolated protoplasts. PEG typically is used in the range from half to an equal volume of the mRNA or protein suspension to be transfected, PEG40% being mostly used in this purpose.

In some cases, the nuclease may be delivered via through biolistic transformation methods or through any other suitable transfection method known in the art (Yoo et al, 2007). In the case of biolistic transformation, the nuclease can be introduced into plant tissues with a biolistic device that accelerates the microprojectiles to speeds of 300 to 600 m/s which is sufficient to penetrate plant cell walls and membranes (see, Klein et al., 1992). Another method for introducing protein or RNA to plants is via the sonication of target cells.

Alternatively, liposome or spheroplast fusion may be used to introduce exogenous material into plants (see, e.g., Christou et al., 1987). Electroporation of protoplasts and whole cells and tissues has also been described (Laursen et al., 1994).

Depending on the method used for transfection and its efficiency, the inventors determined that the optimal protein concentration for performing the methods described herein, especially with TALEN™s, was between 0.01 to 0.1 μg/μl. When using PEG, the volume of the protein suspension was generally between 2 to 20 μl. RNA concentration was found optimal in the range of 1 to 5 μg/μl, it being considered that it can be sometimes advantageous to add non coding RNA, such as tRNA carrier, up to 10 μg/μl, in order to increase RNA bulk. This later adjunction of RNA improves transfection and has a protective effect on the RNA encoding the nuclease with respect to degradative enzymes encountered in the plant cell.

A plant can be obtained by regenerating the plant cell produced by any of the method described herein. When the function of the endogenous gene is suppressed in the plant cell into which the non-silent mutation is introduced at the target DNA site, the phenotype of the plant regenerated from such a plant cell may be changed in association with the suppression of the function of the endogenous gene. Accordingly, the methods described herein make it possible to efficiently perform breeding of a plant. The regeneration of a plant from the plant cell can be carried out by a method known to those skilled in the art which depends on the kind of the plant cell. Examples thereof include the method described in Christou et al. (1997) for transformation of rice species.

In some embodiments, the nuclease can be co-delivered with a plasmid encoding one or more exonuclease proteins to increase sequence-specific nuclease induced mutagenesis efficiency. Such exonucleases include, but are not limited to, members of the Trex family of exonucleases (Therapeutic red cell exchange exonucleases) such as TREX2 (Shevelev et al. 2002). The inventors have surprisingly found that co-delivery of an exonuclease such as TREX with purified I-SceI protein increases the frequency of NHEJ events observed as compared with delivery of the I-SceI protein alone. It is to be noted that other suitable exonucleases may also be used in the methods provided herein.

As used herein the term “identity” refers to sequence identity between two nucleic acid molecules or polypeptides. Identity is determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Alignment algorithms and programs are used to calculate the identity between two sequences. FASTA and BLAST are available as a part of the GCG sequence analysis package (University of Wisconsin, Madison, WI), and are used with default setting. BLASTP may also be used to identify an amino acid sequence having at least 80%, 85%, 87.5%, 90%, 92.5%, 95%, 97.5%, 98%, or 99% sequence similarity to a reference amino acid sequence using a similarity matrix such as BLOSUM45, BLOSUM62 or BLOSUM80. Unless otherwise indicated, a similarity score is based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The same applies with respect to polynucleotide sequences using BLASTN.

As used herein the term “homologous” is intended to mean a sequence with enough identity to another one to lead to a homologous recombination between sequences, more particularly having at least 95% identity (e.g., at least 97% identity, or at least 99% identity).

As used herein the term “endonuclease” refers to an enzyme capable of causing a double-stranded break in a DNA molecule at highly specific locations.

As defined herein the term “exonuclease” refers to an enzyme that works by cleaving nucleotides one at a time from the end (exo) of a polynucleotide chain causing a hydrolyzing reaction that breaks phosphodiester bonds at either the 3′ or the 5′ to occur.

As used herein the term “sequence-specific nuclease” refers to any nuclease enzyme which is able to induce a double-strand DNA break at a desired and predetermined genomic locus

As used herein the term “meganuclease” refers to natural or engineered rare-cutting endonuclease, typically having a polynucleotide recognition site of about 12-40 bp in length, more preferably of 14-40 bp. Typical meganucleases cause cleavage inside their recognition site, leaving 4 nt staggered cut with 3′OH overhangs. The meganuclease are preferably homing endonuclease, more particularly belonging to the dodecapeptide family (LAGLIDADG; SEQ ID NO:55) (WO 2004/067736), TAL-effector like endonuclease, zinc-finger-nuclease, or any nuclease fused to modular base-per-base binding domains (MBBBD)—i.e., endonucleases able to bind a predetermined nucleic acid target sequence and to induce cleavage in sequence adjacent thereto. These meganucleases are useful for inducing double-stranded breaks in specific DNA sequences and thereby promote site-specific homologous recombination and targeted manipulation of genomic sequences.

As used herein the term “vector” refers to a nucleic acid molecule capable of transporting another nucleic acid to which it has been linked into a cell, or a cell compartment.

As used herein the term “zinc finger nuclease” refers to artificial restriction enzymes generated by fusing a zinc finger DNA-binding domain to a DNA-cleavage domain. Briefly, ZFNs are synthetic proteins comprising an engineered zinc finger DNA-binding domain fused to the cleavage domain of the FokI restriction endonuclease. ZFNs may be used to induce double-stranded breaks in specific DNA sequences and thereby promote site-specific homologous recombination and targeted manipulation of genomic sequences.

As used herein the term “TAL-effector endonuclease” refers to artificial restriction enzymes generated by fusing a DNA recognition domain deriving from TALE proteins of Xanthomonas to a catalytic domain of a nuclease, as described by Voytas and Bogdanove in WO 2011/072246. TAL-effector endonucleases are named TALEN™ by the applicant (Cellectis, 8 rue de la Croix Jarry, 75013 PARIS).

The invention will be further described in the following examples, which do not limit the scope of the invention described in the claims.

### EXAMPLES

### Example 1: Designing and Constructing Sequence-Specific Nucleases for Protein Expression

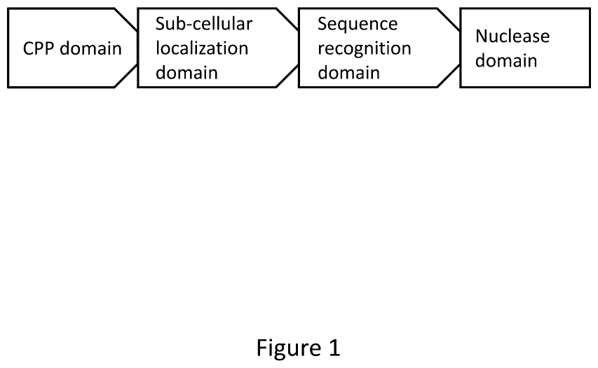

A sequence-specific nuclease typically includes the following components (FIG. 1):
1. A DNA binding domain that recognizes a specific DNA sequence in a plant genome.
2. A nuclease domain that creates a DNA double-strand break at the recognition site in the plant genome. Imprecise repair of the break through non-homologous end-joining introduces mutations at the break site.
3. A sub-cellular localization signal that directs the nuclease to the nucleus, mitochondria or chloroplast.
4. A cell penetration motif that helps the sequence-specific nuclease penetrate cell membranes during transformation.

Sequences encoding the custom nuclease may be synthesized and cloned into a protein expression vector, such as pQE or pET. Functional protein can therefore be expressed in E. coli and purified using standard protocols or commercial kits. Alternatively, other protein expression systems, including yeast, insect or mammalian cells, can be used to produce proteins that are difficult to express and purify in E. coli.

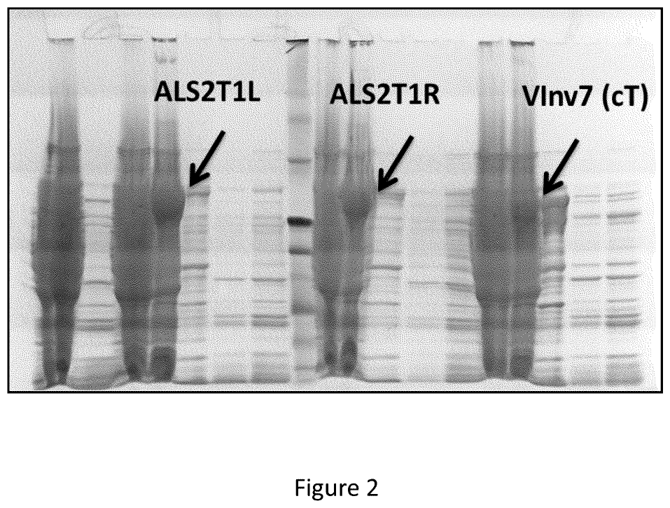

Here, pQE-80L-Kan was used as the protein expression vector. An SV40 nuclear localization signal was added as well as the Tat cell penetration peptide (Frankel and Pabo, 1988, Schwarze et al., 1999). Sequence specific nucleases included a TALEN™ pair targeting a site in the N. benthamiana ALS2 gene. In addition, a compact TALEN™ was also used that targets a site in the VInv7 gene in S. tuberosum. E. coli strain BL21 was used for protein expression (Beurdeley et al., 2013). A Qiagen Ni-NTA Spin kit was used for protein purification. A high yield of recombinant protein was obtained for all three TALEN™ in E. coli (FIG. 2). The plasmids for producing the recombinant TALEN™ were provided by Cellectis Bioresearch (8, rue de la Croix Jarry, 75013 PARIS).

### Example 2: In Vitro Sequence-Specific Nuclease Activity of Purified TALEN™

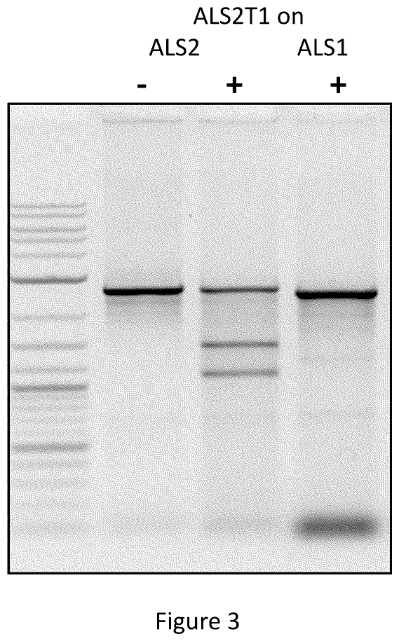

To test the enzyme activity of purified TALEN™, equal amounts of ALS2T1L (SEQ ID NO:2) and ALS2T1R (SEQ ID NO:3) proteins were mixed and incubated with a PCR fragment derived from the N. benthamiana ALS2 gene (the PCR product has the TALEN recognition site). The reaction was carried out at 25° C. and had the following buffer system: 100 mM NaCl, 50 mM Tris-HCl, 10 mM MgCl₂, and 1 mM dithiothreitol, pH 7.9. A PCR fragment derived from the N. benthamiana ALS1 gene (lacking the TALEN recognition site) was used as a negative control. The two TALENs clearly cleaved the ALS2 gene fragment in vitro; no activity was observed with the ALS1 fragment (FIG. 3). The data indicate that the purified TALEN™ have sequence-specific nuclease activity.

### Example 3: Delivery of Sequence-Specific Nucleases to Plant Cells as Proteins

The enzyme I-SceI was purchased from New England Biolabs and dialyzed to remove the buffer supplied by the manufacturer. Briefly, 20 μl (100U) of I-SceI was placed on a Millipore 0.025 μm VSWP filter (CAT#VSWP02500). The filter was floated on a MMG buffer (0.4 M mannitol, 4 mM MES, 15 mM MgCl₂, pH 5.8) at 4° C. for 1 hour. After the enzyme solution was completely equilibrated by MMG buffer, it was transferred to a new tube and kept on ice until further use. The protein was delivered to plant cells by PEG-mediated protoplast transformation. Methods for tobacco protoplast preparation were as previously described (Zhang et al. 2013). Briefly, seeds from a transgenic tobacco line with an integrated I-SceI recognition site were planted in moistened vermiculite and grown under low light conditions for 3-5 weeks (Pacher et al., 2007). Young, fully expanded leaves were collected and surface sterilized, and protoplasts were isolated.

Purified I-SceI protein was introduced into N. tabacum protoplasts by PEG-mediated transformation as described elsewhere (Yoo et al., Nature Protocols 2:1565-1572, 2007). Briefly, 20-200U of I-SceI protein was mixed with 200,000 protoplasts at room temperature in 200 μl of 0.4 M mannitol, 4 mM MES, 15 mM MgCl₂, pH 5.8. Other treatments included transforming protoplasts with DNA that encodes I-SceI (SEQ ID NO:1), DNA that encodes the Trex2 protein, or both. Trex2 is an endonuclease that increases frequencies of imprecise DNA repair through NHEJ (Certo et al. 2012). In addition, in one sample, I-SceI protein was co-delivered with Trex2-encoding DNA (SEQ ID NO:6). After transformation, an equal volume of 40% PEG-4000, 0.2 M mannitol, 100 mM CaCl₂, pH5.8 was added to protoplasts and immediately mixed well. The mixture was incubated in the dark for 30 minutes before washing once with 0.45 M mannitol, 10 mM CaCl₂. The protoplasts were then washed with K3G1 medium twice before moving the cells to 1 ml of K3G1 in a petri dish for long-term culture.

### Example 4: Activity of I-SceI on Episomal Target Sites in N. tabacum

To assess the protein activity of the I-SceI targeting episomal sites in plant cell, a SSA construct was co-delivered with I-SceI protein. For this assay, a target plasmid was constructed with the I-Sce recognition site cloned in a non-functional YFP reporter gene. The target site was flanked by a direct repeat of YFP coding sequence such that if the reporter gene was cleaved by the I-Sce, recombination would occur between the direct repeats and function would be restored to the YFP gene. Expression of YFP, therefore, served as a measure of I-SceI cleavage activity.

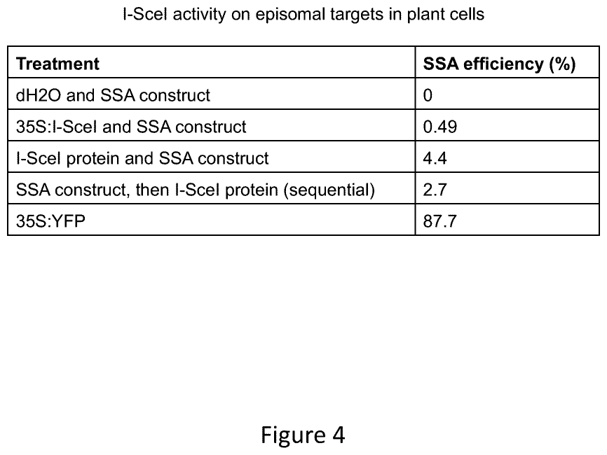

The activity of the I-SceI protein and its control treatments against the episomal target sequence is summarized in FIG. 4. The delivery of I-SceI as DNA yielded a 0.49% YFP expression; while I-SceI protein yielded 4.4% expression of YFP. When the SSA construct and the I-SceI protein were delivered sequentially, the YFP SSA efficiency was 2.4%. The transformation efficiency was indicated by YFP expression of 35S:YFP DNA delivery control.

### Example 5: Activity of I-SceI at Their Endogenous Target Sites in N. tabacum

A transgenic tobacco line contains a single I-SceI recognition site in the genome as described previously (Pacher et al., 2007). Transformed protoplasts isolated from this transgenic line were harvested 24-48 hours after treatment, and genomic DNA was prepared. Using this genomic DNA as a template, a 301 bp fragment encompassing the I-SceI recognition site was amplified by PCR. The PCR product was then subjected to 454 pyro-sequencing. Sequencing reads with insertion/deletion (indel) mutations in the recognition site were considered as having been derived from imprecise repair of a cleaved I-SceI recognition site by NHEJ. Mutagenesis frequency was calculated as the number of sequencing reads with NHEJ mutations out of the total sequencing reads.

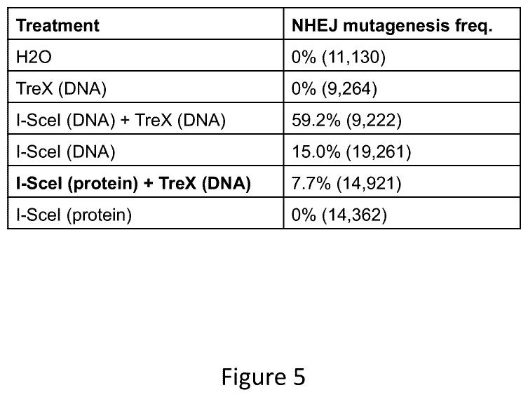

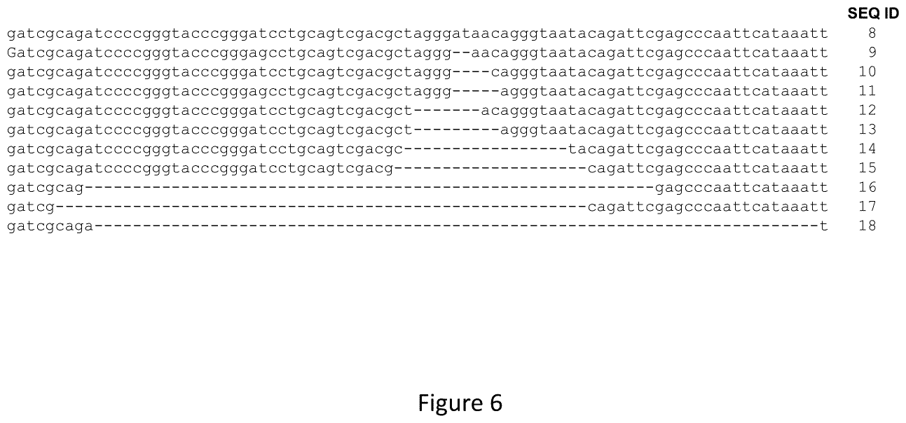

The activity of the I-SceI protein and its control treatments against the target sequence is summarized in FIG. 5. The delivery of I-SceI as DNA (SEQ ID NO:1) yielded a 15% mutagenesis frequency. When combined with DNA encoding the exonuclease Trex2, the mutagenesis frequency increased to 59.2%. When I-SceI was delivered as protein, the mutagenesis activity was undetectable; however, when I-SceI protein was co-delivered with Trex2-encoding DNA (SEQ ID NO:6), a 7.7% mutagenesis frequency was observed. Examples of I-SceI protein induced mutations are shown in FIG. 6. Collectively, the data demonstrate that I-SceI protein creates targeted chromosome breaks when delivered to plant cells as protein. Further, the imprecise repair of these breaks leads to the introduction of targeted mutations.

### Example 6: Delivery of TALEN Proteins to Plant Cells

Purified TALEN™ protein was introduced into N. benthamiana protoplasts by PEG-mediated transformation as described in Example 4. Briefly, 2-20 μl of ALS2T1 protein was mixed with 200,000 protoplasts at room temperature in 200 μl of 0.4 M mannitol, 4 mM MES, 15 mM MgCl₂, pH 5.8. Other treatments included transforming protoplasts with combination of Trex2 protein, DNA that encodes ALS2T1, DNA that encodes the Trex2 protein, or a DNA construct for YFP expression. Trex2 is an endonuclease that increases frequencies of imprecise DNA repair through NHEJ. After transformation, an equal volume of 40% PEG-4000, 0.2 M mannitol, 100 mM CaCl₂, pH 5.8 was added to protoplasts and immediately mixed well. The mixture was incubated in the dark for 30 minutes before washing once with 0.45 M mannitol, 10 mM CaCl₂. The protoplasts were then washed with K3G1 medium twice before moving the cells to 1 ml of K3G1 in a petri dish for long-term culture.

### Example 7: Activity of TALENT ALS2T1 at Their Endogenous Target Sites in N. benthamiana

Transformed protoplasts were harvested 48 hours after treatment, and genomic DNA was prepared. Using this genomic DNA as a template, a 253 bp fragment encompassing the ALS2T1 recognition site was amplified by PCR. The PCR product was then subjected to 454 pyro-sequencing. Sequencing reads with insertion/deletion (indel) mutations in the recognition site were considered as having been derived from imprecise repair of a cleaved I-SceI recognition site by NHEJ. Mutagenesis frequency was calculated as the number of sequencing reads with NHEJ mutations out of the total sequencing reads.

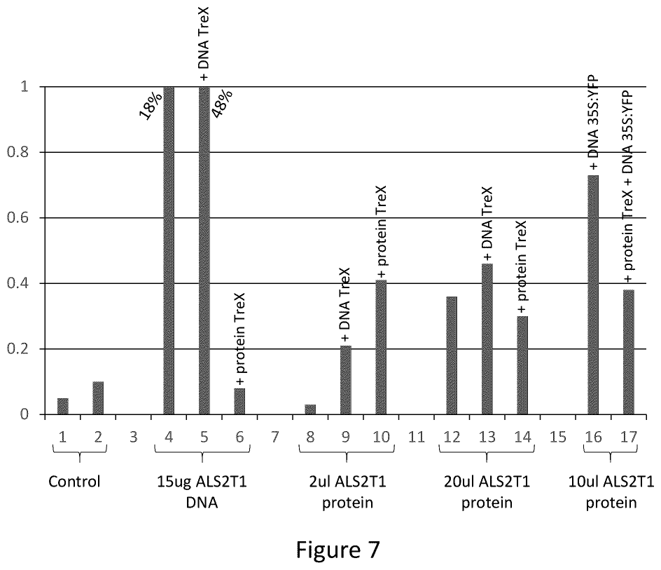

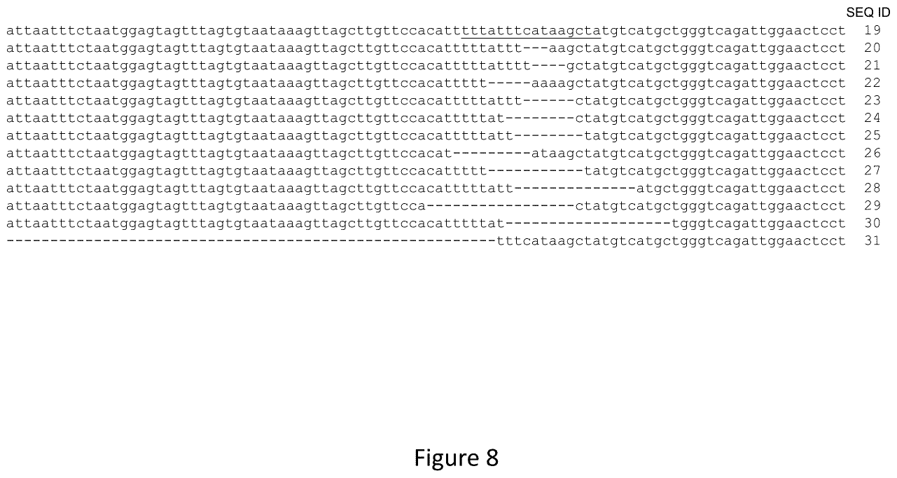

The activity of the ALS2T1 protein and its control treatments against the target sequence is summarized in FIG. 7. The delivery of ALS2T1 as DNA (SEQ ID NOS:2 and 3) yielded an 18.4% mutagenesis frequency. When combined with DNA encoding the exonuclease Trex2 (SEQ ID NO:6), the mutagenesis frequency increased to 48%. When ALS2T1 was delivered as protein, the mutagenesis activity was 0.033% to 0.33%; however, when ALS2T1 protein was co-delivered with 35S:YFP DNA, a 0.72% mutagenesis frequency was observed. Examples of ALS2T1 protein-induced mutations are shown in FIG. 8. Collectively, the data demonstrate that ALS2T1 protein creates targeted chromosome breaks when delivered to plant cells as protein. Further, the imprecise repair of these breaks leads to the introduction of targeted mutations.

### Example 8: Preparation of mRNA Encoding Sequence-Specific Nucleases

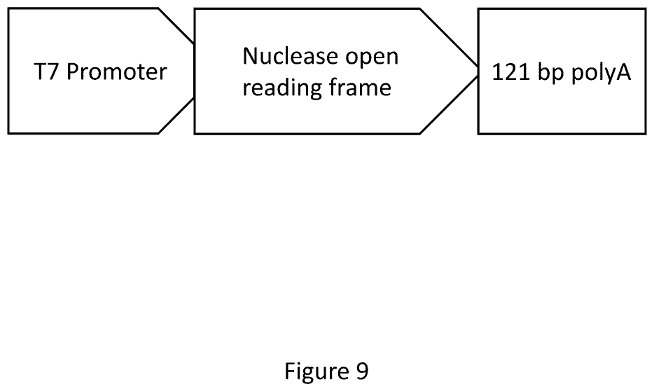

Sequence-specific nucleases, including meganucleases, zinc finger nucleases (ZFNs), or transcription activator-like effector nucleases (TALEN™), are cloned into a T7 expression vector (FIG. 9). Several different 5′ and 3′ UTR pairs were chosen based on data from a genome wide study of transcript decay rates in A. thaliana (Narsai, 2007). The sequences selected were based on the half-lives of various transcripts as well as the functional categories. These UTR pairs were synthesized to allow convenient cloning into the T7-driven plasmid vector. The resulting nuclease constructs are linearized by SapI digestion; a SapI site is located right after the polyA sequences. The linearized plasmid serves as the DNA template for in vitro mRNA production using the T7 Ultra kit (Life Technologies Corporation). Alternatively, mRNA encoding the nuclease can be prepared by a commercial provider. Synthesized mRNAs are dissolved in nuclease-free distilled water and stored at −80° C.

### Example 9: Activity on Episomal Targets of Sequence-Specific Nucleases Delivered as mRNA

A single-strand annealing (SSA) assay was used to measure activity of nuclease mRNAs that had been transformed into tobacco protoplasts (Zhang et al. 2013). As described in Example 4, the SSA assay uses a non-functional YFP reporter that is cleaved by the nuclease. Upon cleavage, recombination between repeated sequences in the reporter reconstitutes a functional YFP gene. YFP fluorescence can then be quantified by flow cytometry.

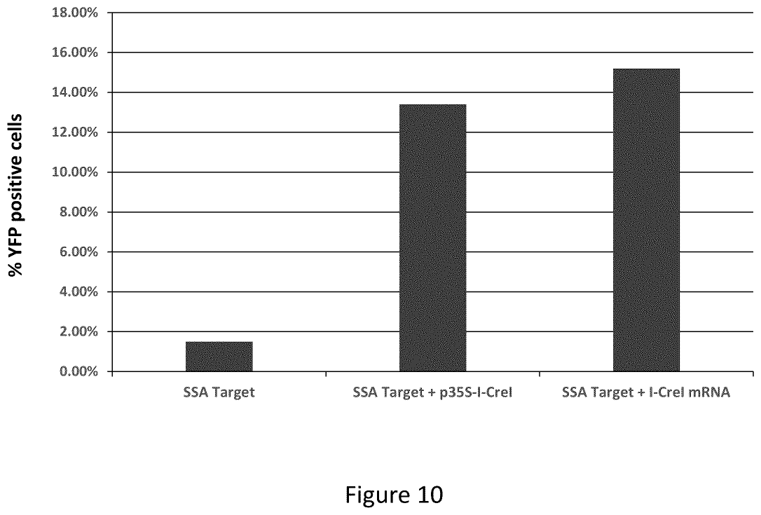

To determine whether mRNA could be delivered to plant cells and mediate targeted DNA modification, I-CreI mRNAs together with a SSA target plasmid were introduced into tobacco protoplasts by PEG-mediated transformation (Golds et al. 1993, Yoo et al. 2007, Zhang et al. 2013). The SSA reporter has an I-CreI site between the repeated sequences in YFP. Methods for tobacco protoplast preparation and transformation were as previously described (Zhang et al. 2013). The SSA target plasmid alone served as a negative control. As a positive control, cells were transformed with a DNA construct expressing I-CreI (p35S-I-CreI) as well as the I-CreI SSA reporter. Twenty-four hours after transformation, YFP fluorescence was measured by flow cytometry (FIG. 10). Similar levels of targeted cleavage of the SSA reporter were observed both with p35S-I-CreI DNA and I-CreI mRNA. The data demonstrate that functional nucleases can be successful delivered to protoplasts in the form of mRNA.

### Example 10: Cleavage Activity on Chromosomal Targets of Sequence-Specific Nucleases Delivered as mRNA

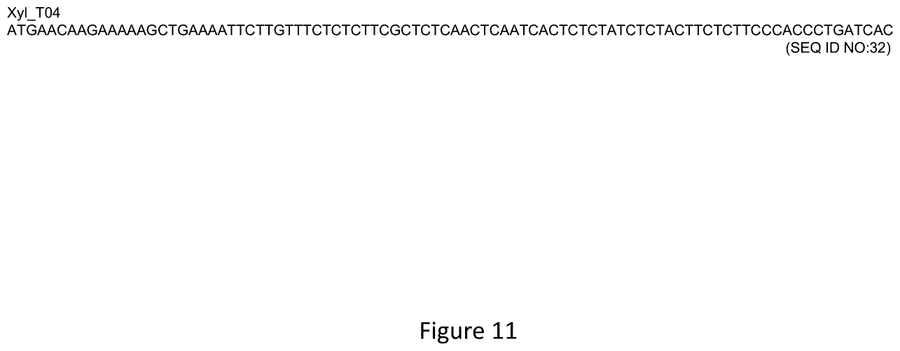

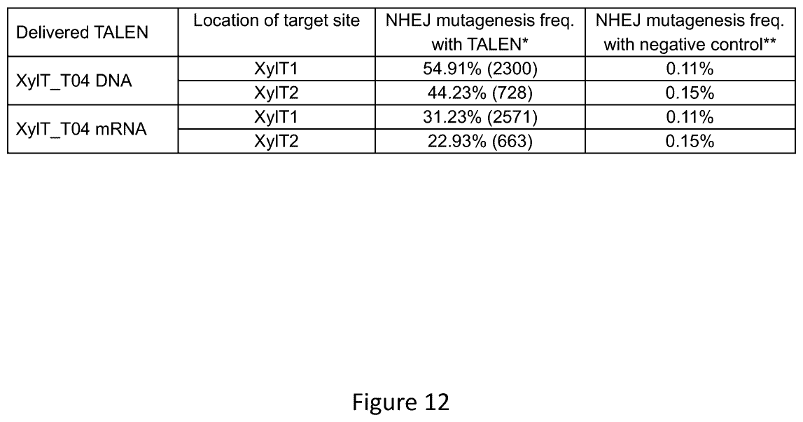

A TALEN pair (XylT TALEN™) was designed to cleave the endogenous β1,2-xylosyltransferase genes of N. benthamiana (Strasser et al. 2008) (FIG. 11). These genes are designated XylT1 and XylT2, and the TALEN™ recognizes the same sequence found in both genes. Each XylT TALEN™ was subcloned into a T7-driven expression plasmid (FIG. 12). The resulting TALEN™ expression plasmids were linearized by SapI digestion and served as DNA templates for in vitro mRNA production as described in Example 8.

TALEN™-encoding plasmid DNA or mRNA were next introduced into N. benthamiana protoplasts by PEG-mediated transformation (Golds et al., 1993, Yoo et al. 2007, Zhang et al., 2013). Protoplasts were isolated from well-expanded leaves of one month old N. benthamiana. The protoplast density was adjusted to the cell density of 5×10⁵/ml to 1×10⁶/ml, and 200 μl of protoplasts were used for each transformation. For mRNA delivery, an RNA cocktail was prepared by mixing 15 μl of L-TALEN mRNA (2 μg/μl), 15 μl of R-TALEN mRNA (2 μg/μl), and 10 μl of yeast tRNA carrier (10 μg/μl). To minimize the potential degradation by RNAse, the RNA cocktail was quickly added to 200 μl of protoplasts, and gently mixed only for a few seconds by finger tapping. Almost immediately, 210 μl of 40% PEG was added, and mixed well by finger tapping for 1 min. The transformation reaction was incubated for 30 min at room temperature. The transformation was stopped by the addition of 900 μl of wash buffer. After a couple of washes, transformed protoplasts were cultured in K3/G1 medium at the cell density of 5×10⁵/ml. The TALEN-encoding plasmid DNA was also transformed as a positive control.

Three days after treatment, transformed protoplasts were harvested, and genomic DNA was prepared. Using the genomic DNA prepared from the protoplasts as a template, an approximately 300-bp fragment encompassing the TALEN™ recognition site was amplified by PCR. The PCR product was then subjected to 454 pyro-sequencing. Sequencing reads with insertion/deletion (indel) mutations in the spacer region were considered as having been derived from imprecise repair of a cleaved TALEN™ recognition site by non-homologous end-joining (NHEJ). Mutagenesis frequency was calculated as the number of sequencing reads with NHEJ mutations out of the total sequencing reads.

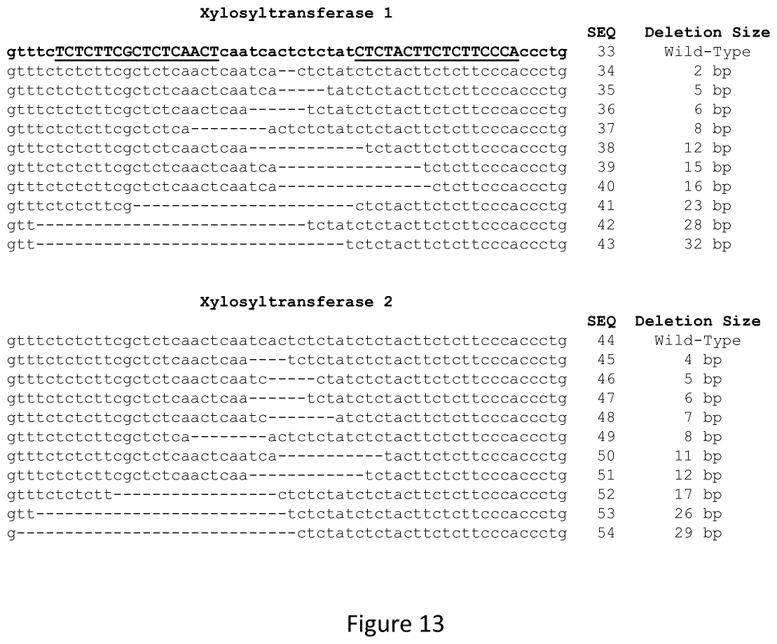

Xyl_T04 TALEN™ DNA and mRNA were tested against their targets, namely the XylT1 and XylT2 genes in N. benthamiana. As indicated above, the TALEN™ recognition sites are present in both XylT1 and XylT2 genes. As summarized in FIG. 12, Xyl_T04 TALEN™ plasmid DNAs induced very high frequencies of NHEJ mutations in both genes, ranging from 31.2% to 54.9%. In parallel, Xyl_T04 TALEN™ mRNAs also induced high frequencies of NHEJ mutations in both genes, ranging from 22.9% to 44.2%. Examples of TALEN™-induced mutations on XylT1 and XylT2 loci are shown in FIG. 13.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### References

Bashirullah A, Cooperstock R, Lipshitz H (2001) Spatial and temporal control of RNA stability. PNAS 98:7025-7028.
Beurdeley M, Bietz F, Li J, Thomas S, Stoddard T, et al. (2013) Compact designer TALEN™ for efficient genome engineering. Nature Communications 4:1762.
Bhushan S, Kuhn C, Berglund AK, Roth C, Glaser E (2006) The role of the N-terminal domain of chloroplast targeting peptides in organellar protein import and miss-sorting. FEBS Letters Volume 580, Issue 16, 10 Jul. 2006, Pages 3966-3972.
Certo M T, Gwiazda K S, Kuhar R, Sather B, Curinga G, et al. (2012) Coupling endonucleases with DNA end-processing enzymes to drive gene disruption. Nature methods 9:973-975. Christou, P. (1997) Rice transformation: bombardment. Plant Mol Biol. 35(1-2):197-203.
Chugh, A., Amundsen, E., Eudes, F. (2009) Translocation of cell-penetrating peptides and delivery of their cargoes in triticale microspores. Plant Cell Rep. 28(5):801-10
Dormann, D., Madl, T., Valori, C. F., Bentmann, E., Tahirovic, S., Abou-Ajram, C., Kremmer, E., Ansorge, O., Mackenzie, I. R., Neumeann, M., Haass, C. (2012) Arginine methylation next to the PY-NLS modulates Transportin binding and nuclear import of FUS. EMBO J. 31(22):4258-75.
Frankel A. D., Pabo C. O. (1988) Cellular uptake of the tat protein from human immunodeficiency virus. Cell 55:1189-1193.
Gaj T., Guo J., Kato Y., Sirk S., Barbas C. (2013) Targeted gene knockout by direct delivery of ZFN proteins. Nat Methods 9(8):805-807
Gasiunas, G., Barrangou, R., Horvath, P., Siksnys, V. (2012) Cas9-crRNA ribonucleoprotein complex mediates specific DNA cleavage for adaptive immunity in bacteria. PNAS 109(39):E2579-86.
Golds T., Maliga P., Koop H. U. (1993) Stable plastid transformation in PEG-treated protoplasts of Nicotiana tabacum. Nature Biotechnology 11:95-97.
Hicks, G. R., Raikhel, N. V. (1993) Specific binding of nuclear localization sequences to plant nuclei. Plant Cell 5(8):983-94
Klein T. M., Arentzen R., Lewis P. A., Fitzpatrick-McElligott S. (1992) Transformation of microbes, plants and animals by particle bombardment. Biotechnology (NY) 10(3):286-91.
Lakshmanan, M., Kodama, Y., Yoshizumi, T., Sudesh, K., Numata, K. (2013) Rapid and efficient delivery into plant cells using designed peptide carriers. Biomacromolecules 14(1):10-6
Laursen C. M., Krzyzek R. A., Flick C. E., Anderson P. C., Spencer T. M., (1994) Production of fertile transgenic maize by electroporation of suspension culture cells. Plant Mol Biol. 24(1):51-61.
Lloyd, D. Plaisier C. L., Carroll D, Drews G. N., (2005) Targeted mutagenesis using zinc-finger nucleases in Arabidopsis. PNAS 102:2232-2237.
Mäe, M., Langel, U. (2006) Cell-penetrating peptides as vectors for peptide, protein and oligonucleotide delivery. Curr. Opin. Pharmacol. 6(5):509-14.
Narsai R., Howell K., Millar A., O'Toole N., Small I., Whelan J. (2007) Genome-Wide Analysis of mRNA Decay Rates and Their Determinants in Arabidopsis thaliana. The Plant Cell 19:3418-3436.
Pacher M., Schmidt-Puchta W., Puchta H. (2007) Two unlinked double-strand breaks can induce reciprocal exchanges in plant genomes via homologous recombination and nonhomologous end joining. Genetics 175:21-29.
Schwarze S. R., Ho A., Vocero-Akbani A., Dowdy S. F. (1999) In vivo protein transduction: delivery of a biologically active protein into the mouse. Science 285:1569-1572.
Shevelev, I. V., Ramadanm K., Hübscher, U. (2002) The TREX2 3′-5′ exonuclease physically interacts with DNA polymerase delta and increases its accuracy. Scientific WorldJournal 2:275-81.
Strasser R., Stadlmann J., Schahs M., Stiegler G., Quendler H., et al. (2008) Generation of glyco-engineered Nicotiana benthamiana for the production of monoclonal antibodies with a homogeneous human-like N-glycan structure. Plant biotechnology journal 6:392-402.
Townsend J. A., Wright D. A., Winfrey R. J., Fu F., Maeder M. L., Joung J. K., Voytas D. F. (2009) High-frequency modification of plant genes using engineered zinc-finger nucleases. Nature 459:442-445.
Wright D. A., Townsend J. A., Winfrey R. J., Jr., Irwin P. A., Rajagopal J., et al. (2005) High-frequency homologous recombination in plants mediated by zinc-finger nucleases. Plant J 44:693-705.
Yoo S. D., Cho Y. H., Sheen J. (2007) Arabidopsis mesophyll protoplasts: a versatile cell system for transient gene expression analysis. Nature Protocols 2:1565-1572.
Zhang Y., Zhang F., Li X, Baller J. A., Qi Y., et al. (2013) Transcription activator-like effector nucleases enable efficient plant genome engineering. Plant Physiology 161:20-27.

What is claimed is:

1. A method for targeted genetic modification of a plant genome without inserting exogenous genetic material into the genome, the method comprising:
(i) providing a plant cell that comprises an endogenous gene to be modified;
(ii) providing a purified Cas9 endonuclease protein and a guide RNA for targeted recognition of the endogenous gene; and
(iii) transfecting the plant cell with said purified Cas9 endonuclease protein and said guide RNA using biolistic or protoplast transformation, such that said Cas9 endonuclease introduces one or more double stranded DNA breaks (DSB) in the genome to produce a plant cell or cells having a detectable targeted genomic modification without the presence of any exogenous Cas9 genetic material in the plant genome.

2. The method of claim 1, wherein said one or more DSBs are repaired by non-homologous end joining (NHEJ).

3. The method of claim 1, wherein introduction of one or more DSBs in the genome is followed by repair of the one or more DSBs through a homologous recombination mechanism.

4. The method of claim 1, wherein the Cas9 endonuclease further comprises one or more subcellular localization domains.

5. The method of claim 4, wherein the one or more subcellular localization domains comprise an SV40 nuclear localization signal, an acidic M9 domain of hnRNPA1, a PY-NLS motif signal, a mitochondrial targeting signal, or a chloroplast targeting signal.

6. The method of claim 1, wherein the Cas9 endonuclease further comprises one or more cell penetrating peptide domains (CPPs).

7. The method of claim 6, wherein said one or more CPPs comprise a transactivating transcriptional activator (Tat) peptide.

8. The method of claim 6, wherein said one or more CPPs comprise a Pep-1 CPP domain.

9. The method of claim 1, wherein the Cas9 endonuclease protein is co-transfected with one or more plasmids encoding one or more exonucleases.

10. The method of claim 9, wherein said one or more exonucleases comprise a member of the TREX exonuclease family.

11. The method of claim 10, wherein the member of the TREX exonuclease family is TREX2.

12. The method of claim 1, wherein said plant cell is from a crop species of alfalfa, barley, bean, corn, cotton, flax, pea, rape, rice, rye, safflower, sorghum, soybean, sunflower, tobacco, or wheat.

13. The method of claim 12, wherein said plant cell is from the genus Nicotiana.

14. The method of claim 12, wherein said plant cell is from the species Arabidopsis thaliana.

15. The method of claim 1, wherein transfection is effected through delivery of said purified Cas9 endonuclease protein into isolated plant protoplasts.

16. The method of claim 1, wherein transfection is effected through delivery of said purified Cas9 endonuclease protein by biolistic transformation.

17. The method of claim 1, further comprising regenerating the plant cell or cells having the detectable targeted genomic modification into a plant.

18. A kit for targeted genetic modification of a plant genome without inserting exogenous genetic material, said kit comprising:
(i) one or more Cas9 proteins;
(ii) one or more plant protoplasts or whole cultured plant cells;
and optionally
(iii) one or more DNA plasmid vectors encoding one or more TREX family exonucleases.


SEQUENCE LISTING (55 sequences)

SEQ ID NO: 1 - DNA, 720 bp, synthetic construct
  source 1..720: mol_type = other DNA
atggccaaaaacatcaaaaaaaaccaggtaatgaacctgggtccgaactctaaactgctgaaagaatacaaatcccagctgatcgaactgaacatcgaacagttcgaagcaggtatcggtctgatcctgggtgatgcttacatccgttctcgtgatgaaggtaaaacctactgtatgcagttcgagtggaaaaacaaagcatacatggaccacgtatgtctgctgtacgatcagtgggtactgtccccgccgcacaaaaaagaacgtgttaaccacctgggtaacctggtaatcacctggggcgcccagactttcaaacaccaagctttcaacaaactggctaacctgttcatcgttaacaacaaaaaaaccatcccgaacaacctggttgaaaactacctgaccccgatgtctctggcatactggttcatggatgatggtggtaaatgggattacaacaaaaactctaccaacaaatcgatcgtactgaacacccagtctttcactttcgaagaagtagaatacctggttaagggtctgcgtaacaaattccaactgaactgttacgtaaaaatcaacaaaaacaaaccgatcatctacatcgattctatgtcttacctgatcttctacaacctgatcaaaccgtacctgatcccgcagatgatgtacaaactgccgaacactatctcctccgaaactttcctgaaagcggccgactaa

SEQ ID NO: 2 - DNA, 2814 bp, synthetic construct
  source 1..2814: mol_type = other DNA
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 3 - DNA, 2832 bp, synthetic construct
  source 1..2832: mol_type = other DNA
atgggcgatcctaaaaagaaacgtaaggtcatcgataaggagaccgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 4 - DNA, 2810 bp, synthetic construct
  source 1..2810: mol_type = other DNA
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgacccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactga

SEQ ID NO: 5 - DNA, 2829 bp, synthetic construct
  source 1..2829: mol_type = other DNA
atgggcgatcctaaaaagaaacgtaaggtcatcgataaggagaccgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactga

SEQ ID NO: 6 - DNA, 2262 bp, Homo sapiens
  source 1..2262: mol_type = genomic DNA
atgggcggggcgcggctcggagcgcgaaacatggcggggcaggacgctggctgcggccgtggcggcgacgactactcagaggacgaaggcgacagcagcgtgtccagggcggctgtggaggtgttcgggaagctgaaggacctaaactgccccttcctcgagggtctgtatatcacagagccaaagacaattcaggaactgctgtgcagcccctcagagtaccgcttggagatcctagagtggatgtgtacccgggtctggccctcactgcaggacaggttcagctcactgaaaggggtcccaacagaggtgaagatccaagaaatgacgaagctgggccacgagctgatgctgtgtgcgccagatgaccaggagctcctcaagggctgtgcctgcgcccagaagcagctacacttcatggaccagttgctcgataccatccggagcctgaccattgggtgctccagttgctcgagcctgatggagcacttcgaggacaccagggagaagaacgaggccttgctgggggagctcttctctagcccccacctgcagatgctcctgaatccagagtgcgacccgtggcccctggacatgcagcccctcctcaacaagcagagtgatgactggcagtgggccagtgcctctgccaagtccgaggaggaggagaagctggcggagcttgccaggcagctgcaggagagtgctgccaagttgcacgcgcttagaacggagtactttgcacagcatgagcaaggggctgctgcgggcgcagccgacatcagcaccctagaccagaagctgcgtctggtcacttccgacttccaccagctaatcttggcttttctccaagtctacgacgacgagctgggcgagtgctgccagcgcccaggccctgacctccacccgtgcggccccatcatccaggccacgcaccagaatctgacttcctacagccaaatccccagaggccaacctaaaaagccggctttagttacgatgactacagttcccacgtgcgcaactctgcccttggctcaaggattccgtgatgttcattttggttttctaagcgagaggctccgagccttccaacctctgactggctggtcctgtgagacccctcgatcagggatgctgctgcaagtggtcatggcagttgctgacacctctgcgaaggccgtggagaccgtgaagaagcagcaaggcgagcagatctgctggggtggcagcagctccgtcatgagtctagctaccaagatgaatgaactaatggagaaatagaaagtcttcagtgatggcctacgccaaagcacaggatggggcgggcaggaagccctctcccaagatcgagttggccgaggatggatgattgtggcagcagaagccgttgcagccccacgttgtgctctaggcagctgggggcgggctgcggccgctgattaaaggccgcctagagcagcctgtgtggcgacaggtgcccagaagcccaggaagccggtcagtgcccgccccagtttgaggacttgctatccccgtgggaacatcaccatgtccgaggcaccccgggccgagacctttgtcttcctggacctggaagccactgggctccccagtgtggagcccgagattgccgagctgtccctctttgctgtccaccgctcctccctggagaacccggagcacgacgagtctggtgccctagtattgccccgggtcctggacaagctcacgctgtgcatgtgcccggagcgccccttcactgccaaggccagcgagatcaccggcctgagcagtgagggcctggcgcgatgccggaaggctggctttgatggcgccgtggtgcggacgctgcaggccttcctgagccgccaggcagggcccatctgccttgtggcccacaatggctttgattatgatttccccctgctgtgtgccgagctgcggcgcctgggtgcccgcctgccccgggacactgtctgcctggacacgctgccggccctgcggggcctggaccgcgcccacagccacggcacccgggcccggggccgccagggttacagcctcggcagcctcttccaccgctacttccgggcagagccaagcgcagcccactcagccgagggcgacgtgcacaccctgctcctgatcttcctgcaccgcgccgcagagctgctcgcctgggccgatgagcaggcccgtgggtgggcccacatcgagcccatgtacttgccgcctgatgaccccagcctggaggcctga

SEQ ID NO: 7 - DNA, 1458 bp, synthetic construct
  source 1..1458: mol_type = other DNA
atgggttccgaggcaccccgggccgagacctttgtcttcctggacctggaagccactgggctccccagtgtggagcccgagattgccgagctgtccctctttgctgtccaccgctcctccctggagaacccggagcacgacgagtctggtgccctagtattgccccgggtcctggacaagctcacgctgtgcatgtgcccggagcgccccttcactgccaaggccagcgagatcaccggcctgagcagtgagggcctggcgcgatgccggaaggctggctttgatggcgccgtggtgcggacgctgcaggccttcctgagccgccaggcagggcccatctgccttgtggcccacaatggctttgattatgatttccccctgctgtgtgccgagctgcggcgcctgggtgcccgcctgccccgggacactgtctgcctggacacgctgccggccctgcggggcctggaccgcgcccacagccacggcacccgggcccggggccgccagggttacagcctcggcagcctcttccaccgctacttccgggcagagccaagcgcagcccactcagccgagggcgacgtgcacaccctgctcctgatcttcctgcaccgcgccgcagagctgctcgcctgggccgatgagcaggcccgtgggtgggcccacatcgagcccatgtacttgccgcctgatgaccccagcctggaggcgactcctccacagaccggtctggatgttccttactccgaggcaccccgggccgagacctttgtcttcctggacctggaagccactgggctccccagtgtggagcccgagattgccgagctgtccctctttgctgtccaccgctcctccctggagaacccggagcacgacgagtctggtgccctagtattgccccgggtcctggacaagctcacgctgtgcatgtgcccggagcgccccttcactgccaaggccagcgagatcaccggcctgagcagtgagggcctggcgcgatgccggaaggctggctttgatggcgccgtggtgcggacgctgcaggccttcctgagccgccaggcagggcccatctgccttgtggcccacaatggctttgattatgatttccccctgctgtgtgccgagctgcggcgcctgggtgcccgcctgccccgggacactgtctgcctggacacgctgccggccctgcggggcctggaccgcgcccacagccacggcacccgggcccggggccgccagggttacagcctcggcagcctcttccaccgctacttccgggcagagccaagcgcagcccactcagccgagggcgacgtgcacaccctgctcctgatcttcctgcaccgcgccgcagagctgctcgcctgggccgatgagcaggcccgtgggtgggcccacatcgagcccatgtacttgccgcctgatgaccccagcctggaggcggccgactga

SEQ ID NO: 8 - DNA, 85 bp, Nicotiana tabacum
  source 1..85: mol_type = genomic DNA
gatcgcagatccccgggtacccgggatcctgcagtcgacgctagggataacagggtaatacagattcgagcccaattcataaatt

SEQ ID NO: 9 - DNA, 83 bp, synthetic construct
  source 1..83: mol_type = other DNA
gatcgcagatccccgggtacccgggagcctgcagtcgacgctagggaacagggtaatacagattcgagcccaattcataaatt

SEQ ID NO: 10 - DNA, 81 bp, synthetic construct
  source 1..81: mol_type = other DNA
gatcgcagatccccgggtacccgggatcctgcagtcgacgctagggcagggtaatacagattcgagcccaattcataaatt

SEQ ID NO: 11 - DNA, 80 bp, synthetic construct
  source 1..80: mol_type = other DNA
gatcgcagatccccgggtacccgggagcctgcagtcgacgctagggagggtaatacagattcgagcccaattcataaatt

SEQ ID NO: 12 - DNA, 78 bp, synthetic construct
  source 1..78: mol_type = other DNA
gatcgcagatccccgggtacccgggatcctgcagtcgacgctacagggtaatacagattcgagcccaattcataaatt

SEQ ID NO: 13 - DNA, 76 bp, synthetic construct
  source 1..76: mol_type = other DNA
gatcgcagatccccgggtacccgggatcctgcagtcgacgctagggtaatacagattcgagcccaattcataaatt

SEQ ID NO: 14 - DNA, 68 bp, synthetic construct
  source 1..68: mol_type = other DNA
gatcgcagatccccgggtacccgggatcctgcagtcgacgctacagattcgagcccaattcataaatt

SEQ ID NO: 15 - DNA, 65 bp, synthetic construct
  source 1..65: mol_type = other DNA
gatcgcagatccccgggtacccgggatcctgcagtcgacgcagattcgagcccaattcataaatt

SEQ ID NO: 16 - DNA, 26 bp, synthetic construct
  source 1..26: mol_type = other DNA
gatcgcaggagcccaattcataaatt

SEQ ID NO: 17 - DNA, 30 bp, synthetic construct
  source 1..30: mol_type = other DNA
gatcgcagattcgagcccaattcataaatt

SEQ ID NO: 18 - DNA, 10 bp, synthetic construct
  source 1..10: mol_type = other DNA
gatcgcagat

SEQ ID NO: 19 - DNA, 96 bp, Nicotiana benthamiana
  source 1..96: mol_type = genomic DNA
attaatttctaatggagtagtttagtgtaataaagttagcttgttccacatttttatttcataagctatgtcatgctgggtcagattggaactcct

SEQ ID NO: 20 - DNA, 93 bp, synthetic construct
  source 1..93: mol_type = other DNA
attaatttctaatggagtagtttagtgtaataaagttagcttgttccacatttttatttaagctatgtcatgctgggtcagattggaactcct

SEQ ID NO: 21 - DNA, 92 bp, synthetic construct
  source 1..92: mol_type = other DNA
attaatttctaatggagtagtttagtgtaataaagttagcttgttccacatttttattttgctatgtcatgctgggtcagattggaactcct

SEQ ID NO: 22 - DNA, 91 bp, synthetic construct
  source 1..91: mol_type = other DNA
attaatttctaatggagtagtttagtgtaataaagttagcttgttccacatttttaaaagctatgtcatgctgggtcagattggaactcct

SEQ ID NO: 23 - DNA, 90 bp, synthetic construct
  source 1..90: mol_type = other DNA
attaatttctaatggagtagtttagtgtaataaagttagcttgttccacatttttatttctatgtcatgctgggtcagattggaactcct

SEQ ID NO: 24 - DNA, 88 bp, synthetic construct
  source 1..88: mol_type = other DNA
attaatttctaatggagtagtttagtgtaataaagttagcttgttccacatttttatctatgtcatgctgggtcagattggaactcct

SEQ ID NO: 25 - DNA, 88 bp, synthetic construct
  source 1..88: mol_type = other DNA
attaatttctaatggagtagtttagtgtaataaagttagcttgttccacatttttatttatgtcatgctgggtcagattggaactcct

SEQ ID NO: 26 - DNA, 87 bp, synthetic construct
  source 1..87: mol_type = other DNA
attaatttctaatggagtagtttagtgtaataaagttagcttgttccacatataagctatgtcatgctgggtcagattggaactcct

SEQ ID NO: 27 - DNA, 85 bp, synthetic construct
  source 1..85: mol_type = other DNA
attaatttctaatggagtagtttagtgtaataaagttagcttgttccacattttttatgtcatgctgggtcagattggaactcct

SEQ ID NO: 28 - DNA, 82 bp, synthetic construct
  source 1..82: mol_type = other DNA
attaatttctaatggagtagtttagtgtaataaagttagcttgttccacatttttattatgctgggtcagattggaactcct

SEQ ID NO: 29 - DNA, 79 bp, synthetic construct
  source 1..79: mol_type = other DNA
attaatttctaatggagtagtttagtgtaataaagttagcttgttccactatgtcatgctgggtcagattggaactcct

SEQ ID NO: 30 - DNA, 77 bp, synthetic construct
  source 1..77: mol_type = other DNA
attaatttctaatggagtagtttagtgtaataaagttagcttgttccacatttttattgggtcagattggaactcct

SEQ ID NO: 31 - DNA, 40 bp, synthetic construct
  source 1..40: mol_type = other DNA
tttcataagctatgtcatgctgggtcagattggaactcct

SEQ ID NO: 32 - DNA, 90 bp, Nicotiana benthamiana
  source 1..90: mol_type = genomic DNA
atgaacaagaaaaagctgaaaattcttgtttctctcttcgctctcaactcaatcactctctatctctacttctcttcccaccctgatcac

SEQ ID NO: 33 - DNA, 58 bp, Nicotiana benthamiana
  source 1..58: mol_type = genomic DNA
gtttctctcttcgctctcaactcaatcactctctatctctacttctcttcccaccctg

SEQ ID NO: 34 - DNA, 56 bp, synthetic construct
  source 1..56: mol_type = other DNA
gtttctctcttcgctctcaactcaatcactctatctctacttctcttcccaccctg

SEQ ID NO: 35 - DNA, 53 bp, synthetic construct
  source 1..53: mol_type = other DNA
gtttctctcttcgctctcaactcaatcatatctctacttctcttcccaccctg

SEQ ID NO: 36 - DNA, 52 bp, synthetic construct
  source 1..52: mol_type = other DNA
gtttctctcttcgctctcaactcaatctatctctacttctcttcccaccctg

SEQ ID NO: 37 - DNA, 50 bp, synthetic construct
  source 1..50: mol_type = other DNA
gtttctctcttcgctctcaactctctatctctacttctcttcccaccctg

SEQ ID NO: 38 - DNA, 46 bp, synthetic construct
  source 1..46: mol_type = other DNA
gtttctctcttcgctctcaactcaatctacttctcttcccaccctg

SEQ ID NO: 39 - DNA, 43 bp, synthetic construct
  source 1..43: mol_type = other DNA
gtttctctcttcgctctcaactcaatcatctcttcccaccctg

SEQ ID NO: 40 - DNA, 42 bp, synthetic construct
  source 1..42: mol_type = other DNA
gtttctctcttcgctctcaactcaatcactcttcccaccctg

SEQ ID NO: 41 - DNA, 35 bp, synthetic construct
  source 1..35: mol_type = other DNA
gtttctctcttcgctctacttctcttcccaccctg

SEQ ID NO: 42 - DNA, 30 bp, synthetic construct
  source 1..30: mol_type = other DNA
gtttctatctctacttctcttcccaccctg

SEQ ID NO: 43 - DNA, 26 bp, synthetic construct
  source 1..26: mol_type = other DNA
gtttctctacttctcttcccaccctg

SEQ ID NO: 44 - DNA, 58 bp, Nicotiana benthamiana; identical to SEQ ID NO: 33
  source 1..58: mol_type = genomic DNA

SEQ ID NO: 45 - DNA, 54 bp, synthetic construct
  source 1..54: mol_type = other DNA
gtttctctcttcgctctcaactcaatctctatctctacttctcttcccaccctg

SEQ ID NO: 46 - DNA, 53 bp, synthetic construct
  source 1..53: mol_type = other DNA
gtttctctcttcgctctcaactcaatcctatctctacttctcttcccaccctg

SEQ ID NO: 47 - DNA, 52 bp, synthetic construct; identical to SEQ ID NO: 36
  source 1..52: mol_type = other DNA

SEQ ID NO: 48 - DNA, 51 bp, synthetic construct
  source 1..51: mol_type = other DNA
gtttctctcttcgctctcaactcaatcatctctacttctcttcccaccctg

SEQ ID NO: 49 - DNA, 50 bp, synthetic construct; identical to SEQ ID NO: 37
  source 1..50: mol_type = other DNA

SEQ ID NO: 50 - DNA, 47 bp, synthetic construct
  source 1..47: mol_type = other DNA
gtttctctcttcgctctcaactcaatcatacttctcttcccaccctg

SEQ ID NO: 51 - DNA, 46 bp, synthetic construct; identical to SEQ ID NO: 38
  source 1..46: mol_type = other DNA

SEQ ID NO: 52 - DNA, 41 bp, synthetic construct
  source 1..41: mol_type = other DNA
gtttctctcttctctctatctctacttctcttcccaccctg

SEQ ID NO: 53 - DNA, 32 bp, synthetic construct
  source 1..32: mol_type = other DNA
gtttctctatctctacttctcttcccaccctg

SEQ ID NO: 54 - DNA, 29 bp, synthetic construct
  source 1..29: mol_type = other DNA
gctctatctctacttctcttcccaccctg

SEQ ID NO: 55 - protein, 9 aa, synthetic construct
LAGLIDADG